SEQUENCE LISTING (5 sequences)

SEQ ID NO: 1 - protein, 343 aa, Artificial sequence
  Synthetic polypeptide
MAEIYNKDGNKLDLYGKIDGLHYFSDDKSVDGDQTYMRVGVKGETQINDQLTGYGQWEYNVQANNTESSSNQAWTRLAFAGLKFGDAGSFDYGRNYGVVYDVTSWTDVLPEFGGDTYGSDNFLQSRANGVATYRNSDFFGLVDGLNFALQYQGKNGSVSGEGATNNGRGAQKQNGDGFGTSVTYDIFDGISAGFAYSHSKRTDDQNNLVLGNGDNAETYTGGLKYDANNIYLATQYTQTYNATRAGSLGFANKAQNFEVVAQYQFDFGLRPSVAYLQSKGKDLEGYGDQDILKYVDVGATYYFNKNMSTYVDYKINLLDDNSFTRNAGVSTDDVVALGLVYQF

SEQ ID NO: 2 - protein, 190 aa, Artificial sequence
  Synthetic polypeptide
MAGEFFIRAGTATVRPTEGSDNVLGSLGSFNVSNNTQLGLTFTYMATDNIGVELLAATPFRHKVGTGPTGTIATVHQLPPTLMAQWYFGDAQSKVRPYVGAGINYTTFFNENFNDTGKAAGLSDLSLKDSWGAAGQVGLDYLINRDWLLNMSVWYMDIDTDVKFKANGVDQKVSTRLDPWVFMFSAGYRF

SEQ ID NO: 3 - protein, 187 aa, Artificial sequence
  Synthetic polypeptide
MCSLHTPQGPGKSPDSPQWRQHQQAVRSLNQFQTRGAFAYLSDEQKVYARFFWQQTGQDRYRLLLTNPLGSTELSLTAQPGSVQLIDNKGQTYTAADAEEMIGRLTGMPIPLNSLRQWIIGLPGDATDYSLDDRYRLRELNYTQNGKTWHVTYGGYTSDTQPALPSNVELNNGAQRIKLKMDNWIVK

SEQ ID NO: 4 - protein, 345 aa, Artificial sequence
  Synthetic polypeptide
MAEIYNKDGNKLDLYGKIDGLHYFSDDKSVDGDQTYMRVGVKGETQINDQLTGYGQWEYNVQANNTESSSDQAWTRLAFAGLKFGDAGSFDYGRNYGVVYDVTSWTDVLPEFGGDTYGSDNFLQSRANGVATYRNSDFFGLVDGLNFALQYQGKNGSVSGEGATNNGRGWSKQNGDGFGTSLTYDIWDGISAGFAYSHSKRTDEQNSVPALGRGDNAETYTGGLKYDANNIYLASQYTQTYNATRAGSLGFANKAQNFEVVAQYQFDFGLRPSVAYLQSKGKDLERGYGDQDILKYVDVGATYYFNKNMSTYVDYKINLLDDNSFTRNAGISTDDVVALGLVYQF

SEQ ID NO: 5 - protein, 171 aa, Artificial sequence
  Synthetic polypeptide
MNKIARLSALAVVLAASVGTTAFAATSTVTGGYAQSDMQGKANKAGGFNLKYRYEQDNNPLGVIGSFTYTEKDNTSNGTYNKGQYYGITAGPAYRLNDWASIYGVVGVGYGKFQNNNFPNQKSDMSDYGFSYGAGLQFNPIENVALDFSYEQSRIRNVDVGTWIAGVGYRF

(19) United States
(12) Patent Application Publication
(10) Pub. No.: US 2022/0160857 A1
(43) Pub. Date: May 26, 2022

(54) MULTIVALENT VACCINES DERIVED FROM KLEBSIELLA OUT MEMBRANE PROTEINS

(71) Applicants: The Administrators of the Tulane Educational Fund, New Orleans, LA (US); University of Pittsburgh-Of the Commonwealth System of Higher Education, Pittsburgh, PA (US)

(72) Inventors: Jay KOLLS, New Orleans, LA (US); Elizabeth NORTON, New Orleans, LA (US); Kong CHEN, Pittsburgh, PA (US)

(73) Assignees: The Administrators of the Tulane Educational Fund, New Orleans, LA (US); University of Pittsburgh-Of the Commonwealth System of Higher Education, Pittsburgh, PA (US)

(21) Appl. No.: 17/601,051

(22) PCT Filed: Mar. 27, 2020
(86) PCT No.: PCT/US2020/025518

§ 371 Date: Oct. 1, 2021

Related U.S. Application Data
(60) Provisional application No. 62/828,211, filed on Apr. 2, 2019.

(51) Int. Cl. A61K 39/108; A61P 31/04

(52) U.S. Cl. CPC A61K 39/0266; A61K 2039/55544; A61P 31/04

(57) ABSTRACT

The present disclosure describes multivalent antibacterial vaccines for the treatment of bacterial infections, such as Klebsiella, E. coli and E. cloacae, as well as therapy-resistant forms thereof.

### PRIORITY CLAIM

This application claims benefit of priority to U.S. Provisional Application Ser. No. 62/828,211, filed Apr. 2, 2019, the entire contents of which are hereby incorporated by reference.

### STATEMENT OF GOVERNMENT SUPPORT

The invention was made with government support under Grant Nos. R37HL079142 and R35HL139930 awarded by the National Institutes of Health. The government has certain rights in the invention.

### BACKGROUND

### 1. Field

This disclosure relates to the fields of microbiology, immunology and medicine. More particularly, the disclosure relates to multivalent broad-spectrum vaccines against various bacterial species, including Klebsiella, derived from outer membrane proteins.

### 2. Related Art

Anti-microbial resistance in Gram-negative bacteria is significant health issue in the U.S. This has been illustrated with significant outbreaks of infection with carbapenemases expressing strains of K. pneumoniae at the NIH Clinical Center (Satline et al., Antimicrob. Agents Chemother. 61(4), 2017) and other hospitals throughout the Country (Satlin et al., Clin. Infect. Dis. 64(7)839-844, 2017). The inventors were first to show that vaccines eliciting tissue specific memory Th17 cells could provide serotype-independent immunity to several serotypes of K. pneumoniae including the New Delhi metallolactamase (NDM1) strain (Chen et al., Immunity 35(6):997-1000, 2011).

In collaboration with the NIH, the inventors generated recombinant outer membrane protein X (OmpX) and showed that this antigen could be used to generate patient specific T-cells ex vivo for potential T-cell based cell therapy. Furthermore, they shown that outer membrane vesicles derived from K2 K. pneumoniae can also provide serotype-independent protection to a hypervirulent K1 strain. In addition, they have shown that OmpX as a subunit vaccine adjuvanted with LTA1 or dmLT-derived from E. coli labile toxin have efficacy with either subcutaneous administration to mucosal (intranasal) administration. The latter route was associated with local type 17 responses in the lung that they have recently showed can regulate chemokine gradients, particularly CXCL5, in lung epithelial cells (Chen et al., Immunity 35(6):997-1000, 2011). Nonetheless, improved vaccines with broad application to drug-resistant bacteria are needed.

### SUMMARY

Thus, in accordance with the present disclosure, there is provided a composition comprising three or more outer membrane proteins (OMPs) from Klebsiella and at least one adjuvant dispersed in a pharmaceutically acceptable buffer, diluent or excipient. The composition may comprise three or all four more of OmpC, OmpW, Omplolb and Omp36K. The composition may comprise adjuvants selected from one or both of LTA1 and/or dmLT. The adjuvant may be linked to one or more of said OMPs. The composition may further comprise OmpX. The composition may be formulated for intranasal administration, for subcutaneous administration, for sublingual or for intramuscular administration. The composition may comprise OmpC, OmpW, Omplolb, Omp36K, LTA1 and dmLT. The composition may comprise OmpC, OmpW, Omplolb, Omp36K, LTA1 and dmLT, but does not include any other OMPs or adjuvants.

Also provided is a method of generating an immune response to a bacterium in a subject comprising administering to said subject a composition comprising three or more outer membrane proteins (OMPs) from Klebsiella and at least one adjuvant dispersed in a pharmaceutically acceptable buffer, diluent or excipient. The composition may comprise three or all four more of OmpC, OmpW, Omplolb and Omp36K. The composition may comprise adjuvants selected from one or both of LTA1 and/or dmLT. The adjuvant may be linked to one or more of said OMPs. The composition may further comprise OmpX. The composition may be administered via intranasal administration, subcutaneous administration, sublingual or intramuscular administration. The composition may comprise OmpC, OmpW, Omplolb, Omp36K, LTA1 and dmLT. The composition may comprise OmpC, OmpW, Omplolb, Omp36K, LTA1 and dmLT, but does not include any other OMPs or adjuvants. The bacteria may be Klebsiella, such as Klebsiella pneumoniae or Klebsiella oxytoca, or may be Escherichia coli or Enterobacter cloacae. The bacteria maybe multi-drug resistant. The subject may have a nosocomial bacterial infection, a post-surgical bacterial infection, a wound bacterial infection, and/or a chronic or persistent bacterial infection.

Another embodiment comprises a method of treating or preventing a bacteria infection in a subject comprising administering to said subject a composition comprising three or more outer membrane proteins (OMPs) from Klebsiella and at least one adjuvant dispersed in a pharmaceutically acceptable buffer, diluent or excipient. The composition may comprise three or all four more of OmpC, OmpW, Omplolb and Omp36K. The composition may comprise adjuvants selected from one or both of LTA1 and/or dmLT. The adjuvant may be linked to one or more of said OMPs. The composition may further comprise OmpX. The composition may be administered via intranasal administration, subcutaneous administration, sublingual or intramuscular administration. The composition may comprise OmpC, OmpW, Omplolb, Omp36K, LTA1 and dmLT. The composition may comprise OmpC, OmpW, Omplolb, Omp36K, LTA1 and dmLT, but does not include any other OMPs or adjuvants.

The bacterial infection may be Klebsiella, such as Klebsiella pneumoniae or Klebsiella oxytoca, or Escherichia coli or Enterobacter cloacae. The bacterial infection may be multi-drug resistant. The subject may have a nosocomial bacterial infection, a post-surgical bacterial infection, or a wound bacterial infection. The subject may have a chronic or persistent bacterial infection. The method may further comprise treating said subject with another anti-bacterial therapy, such as an antibiotic. The other anti-bacterial therapy may be given before and/or after said composition, or concurrent with said composition.

The subject may be a human subject or a non-human mammal. The non-human mammal may be a cow, such as a cow suffering from bovine mastitis.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE FIGURES

The following drawings form part of the present specification and are included to further demonstrate certain aspects of the present disclosure. The disclosure may be better understood by reference to one or more of these drawings in combination with the detailed.

FIG. 1—K. pneumoniae vaccine efficacy. Left panel: Lung CFU 20 hours after bacterial challenge with a K1 strain of K. pneumoniae in female C57Bl/6 mice immunized subcutaneously with vehicle, 100 ng OmpX (derived from serotype 2 K. pneumoniae) or 100 ng OmpX with dmLT or LTA1. Right panel: Lung CFU of K1 strain of K. pneumoniae 20 hrs after bacterial challenge in female C57Bl/6 mice immunized intranasally with vehicle, 10 μg OMVs, 100 ng OmpX with dmLT or LTA1. N=6-7 per group, * p<0.05, ANOVA with multiple comparisons correction. Note that a vaccine derived from a K2 strain can provide immunity to a K1 strain due to the fact that OmpX is conserved across strains.

FIG. 2—Vaccine study histology. The mucosal deliver of the vaccine with adjuvant results in the development of mucosal tertiary lymphoid follicles which mediate local antibodies and T cell responses that mediate the protection by the vaccine. Lymphocyte infiltration, suggesting iBALT, was observed around the bronchus of the OmpX LTA1 mice.

FIG. 3—K. pneumoniae-specific IgG are induced by OmpX vaccination. Vaccination with OmpX-derived from K2 (strain 43816 K. pneumoniae) elicits antibodies that cross-react with a K1 396 strain as well as the multi-drug resistant ST258 strain of K. pneumoniae.

FIG. 4—Quadrivalent K. pneumoniae vaccine experiment. Experimental design to test the quadrivalent vaccine.

FIG. 5—K. pneumoniae vaccine experiment #3. To build upon the monovalent OmpX vaccine, the inventors developed a quadravalent vaccine containing OmpC, OmpW, Omplolb, and Omp36k. These antigens complexed with adjuvant are superior to the monovalent OMPx vaccine in conferring protection against a challenge with K1 K. pneumoniae. Lung and spleen CFU are significantly decreased by the vaccination qith quadrivalent OMPs. Data represent the mean±SEM (n=5). Significant differences are designated by using ANOVA (Kruskall Wallis test) followed by Dunn's multiple comparisons test.

FIG. 6—K. pneumoniae vaccine experiment #3 (IL17A ELISPOT). All components of the vaccine are immunogenic and result in IL-17 T cell responses as assayed by ELISPOT.

FIG. 7—K. pneumoniae vaccine experiment. Vaccine design to determine if the vaccine can work independent of B cells.

FIG. 8—Vaccine experiment of uMT mice (CFU). Although antibodies are generated the maintains efficacy in the lung in mice with genetic deletion of B cells. Thus, this vaccine generates both T and B cell responses and thus could be effective in pateints that are B cell or T cell immunosuppression. Data represent each value and the mean (n=5). Significant differences are designated by using ANOVA followed by Dunn's multiple comparisons test. *, P<0.05, **, P<0.01, ***, P<0.001, ****, P<0.0001.

FIG. 9—Vaccine experiment of IL17rcPostE2Acre mice (CFU). Vaccine efficacy in the lung requires the receptor for IL-17-IL-17RC suggesting that the vaccine is working through the generation of vaccine specific T cells at the mucosa. However, protection in the spleen is maintained which is likely due to the generation of antibody. The effect of vaccine was not completely diminished in IL17rcKO mice, especially in the spleen. Data represent each value and the mean (n=5). Significant differences are designated by using ANOVA followed by Dunn's multiple comparisons test. *, P<0.05, **, P<0.01.

FIG. 10—Amino acid sequences for vaccine components.

FIGS. 11A-B—Antigen specific IgA. (FIG. 11A) 6-8 week old male DermoCre^{-/-} x Il17ra^{fl/fl} (Il17ra^{fl/fl}), and DermoCre^{-/+}x Il17ra^{fl/fl} (Il17ra^{ΔDermo}) mice were immunized with serotype 2 OmpX (1 μg) with LTA1 (10 μg) twice at 3 weeks intervals. 6-8 week old male unimmunized C57Bl/6 mice were used as controls. One week after the last immunization, mice were challenged with a heterologous K1 serotype and CFU in lungs were measured 24 hours post challenge. Significant differences in lung CFU were confirmed by using Kruskal-Wallis and multiple comparisons testing. (FIG. 11B) 6-8 week old male DermoCre^{-/-} x Il17ra^{fl/fl} (Il17ra^{fl/fl}), and DermoCre^{-/+}x Il17ra^{fl/fl} (Il17ra^{ΔDermo}) mice were immunized with serotype 2 OmpX (1 ug) with LTA1 (10 μg) twice at 3 weeks intervals. One week after the last immunization, these mice were euthanized. 6-8 week old male unimmunized C57Bl/6 mice were used as controls. BAL was performed using cold 1 ml PBS and the supernatants following the centrifuge (400×g, 7 min) were collected. Significant differences in OmpX IgA are designated by calculating the AUC and using ANOVA followed by Tukey's multiple comparisons test.

FIG. 12—Based on the conservation of OmpX in members of the Enterobacteriaceae family, the inventors predicted that immunization with Ompx from serotype 2 K. pneumoniae would elicit cross-reactive T cells with other K. pneumoniae serotypes as well as other members of the Enterobacteriaceae family such as E. coli. To test this, they immunized mice with OmpX+LTA1 adjuvant twice, three weeks apart and then assessed the ability of these cells to produce IL-17 in response ex vivo using an Elispot assay. As shown in FIG. 12, mice that were vaccinated with Ompx from K2 produced IL-17 (denoted by gray color) when stimulated with heat killed strains of K. pneumoniae including two carbapenem resistant strains (ST258 C4 and ST258 Il) as well as E. coli. Thus this vaccine elicits immune responses to a broad class of gram negative pathogens.

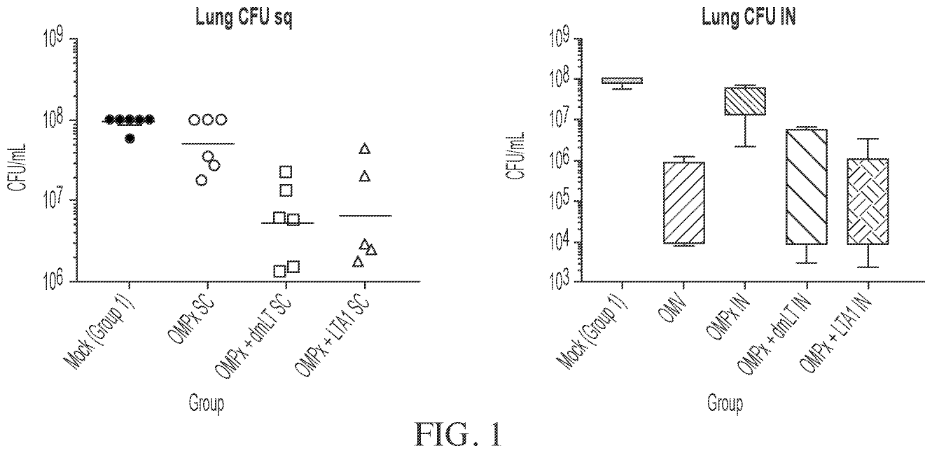

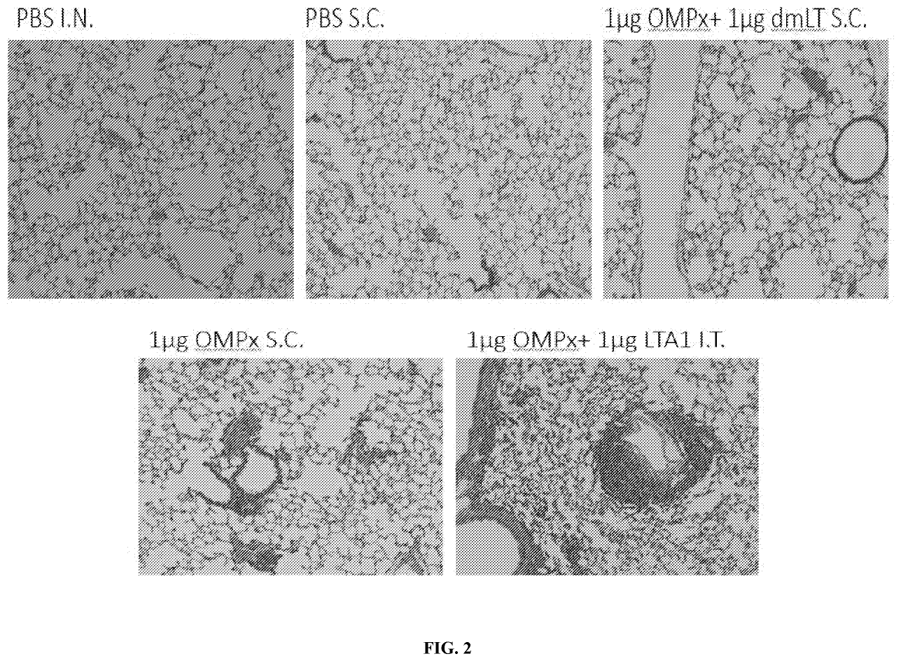

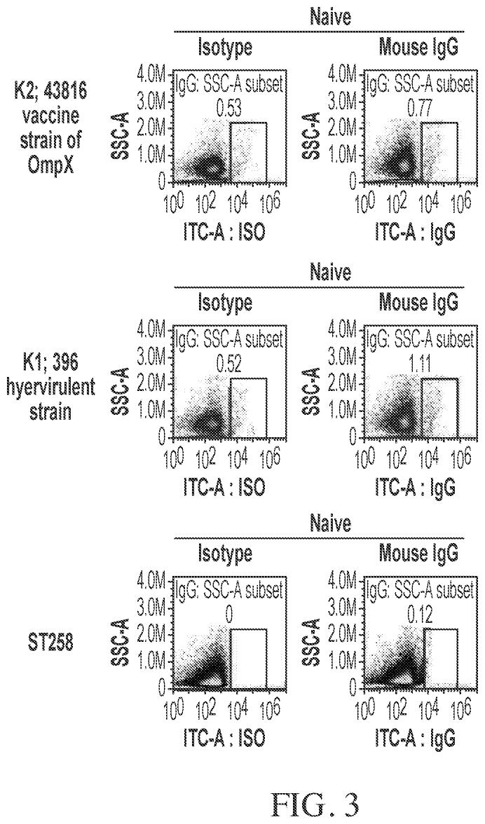

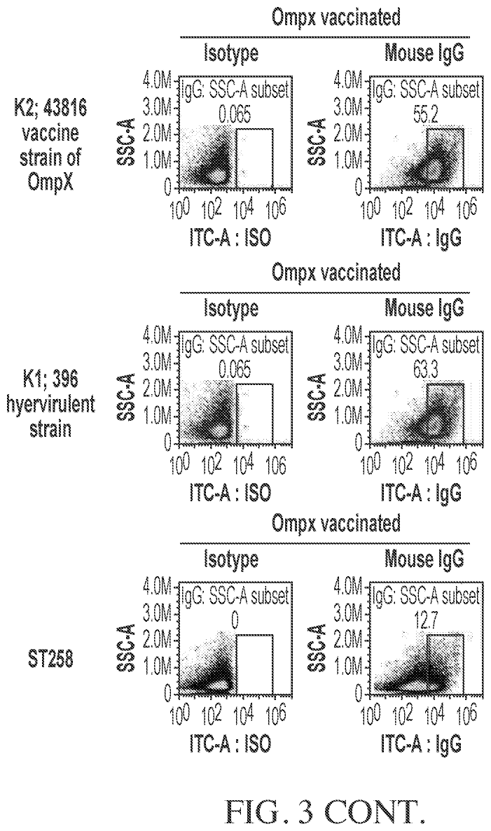

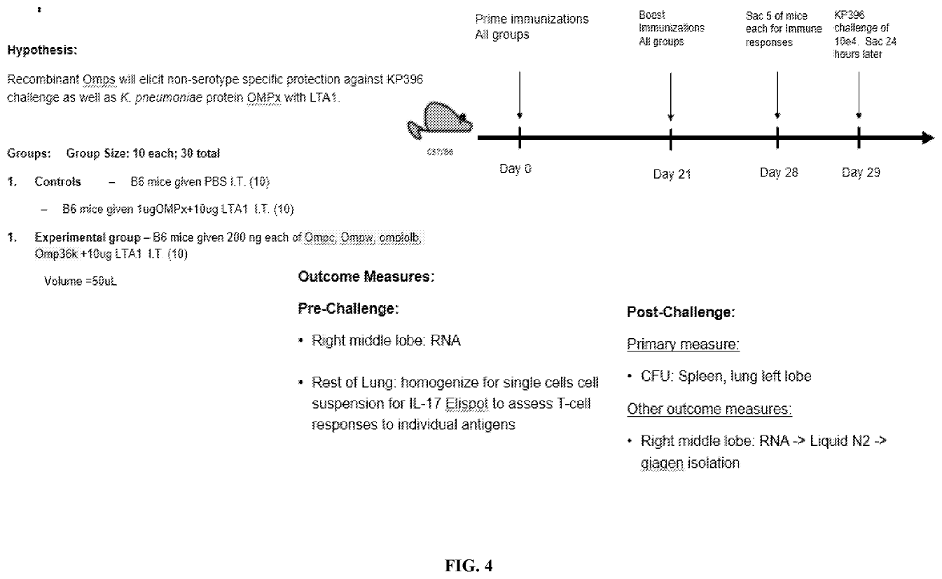

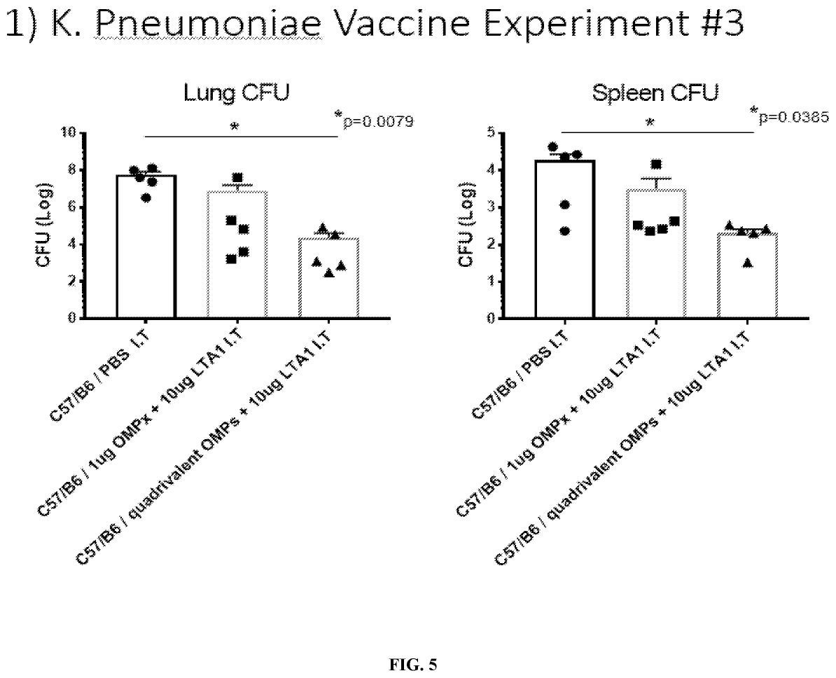

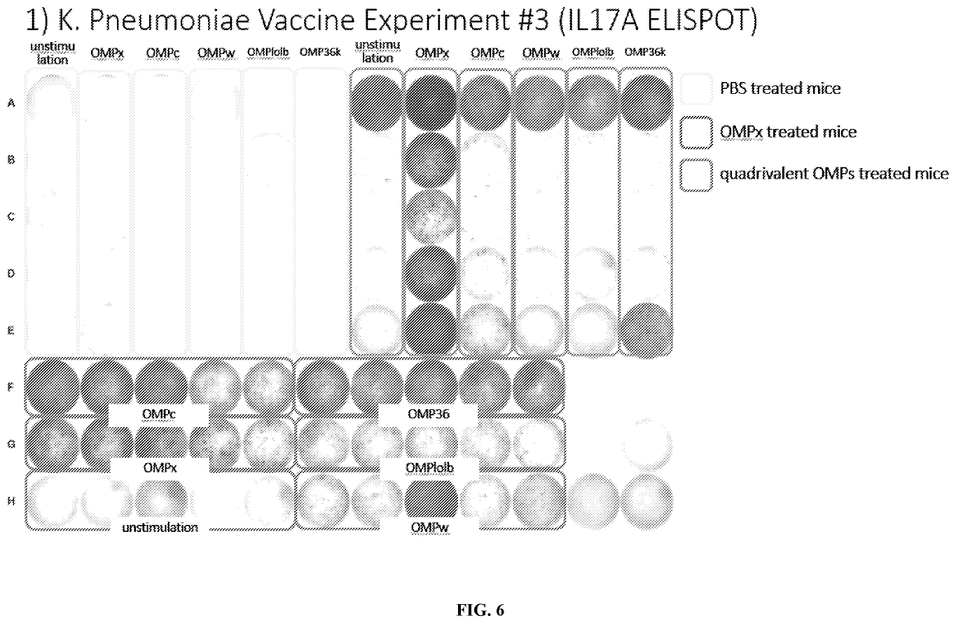

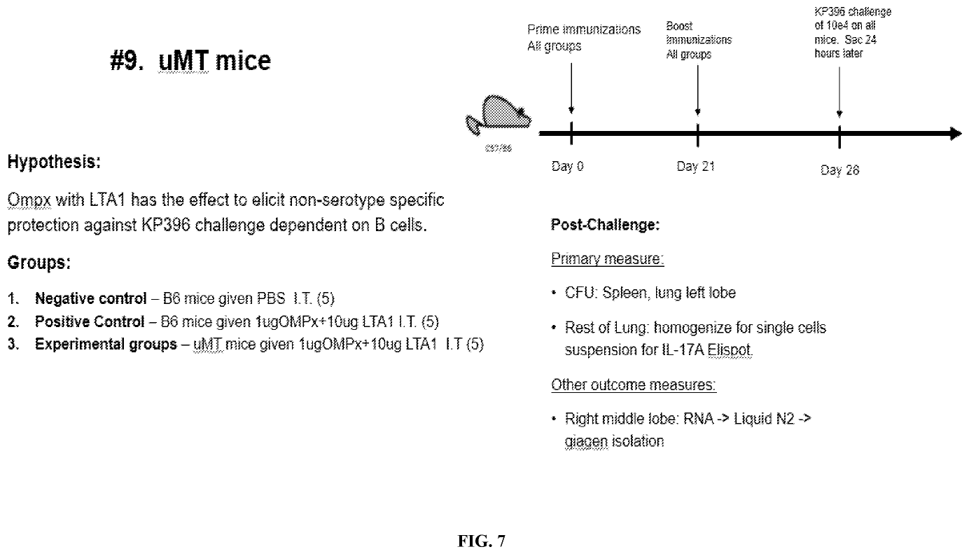

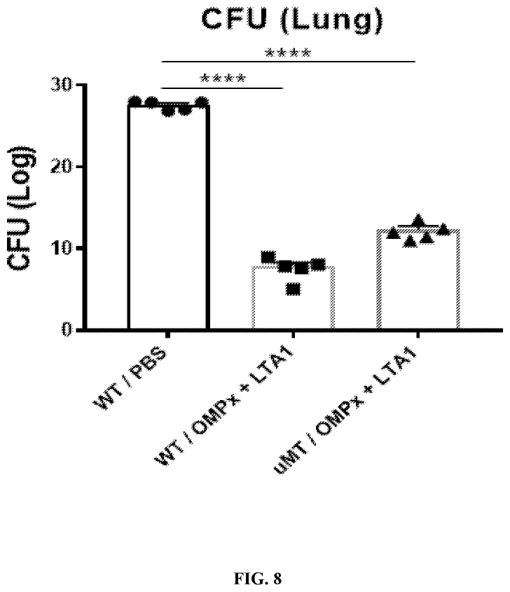

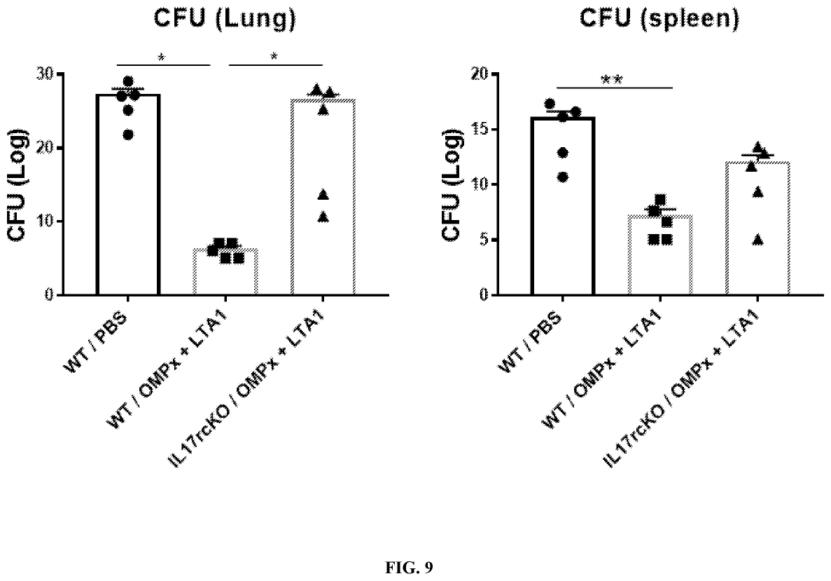

### DESCRIPTION OF ILLUSTRATIVE EMBODIMENTS

As discussed above, drug resistant bacterial infections continue to be a major health concern around the world. The inventors have previously shown that Th17 cells elicited by heat-killed K. pneumoniae proliferate in response to outer membrane proteins (Chen et al., Immunity 35(6):997-1000, 2011). As these proteins can be packaged into outer membrane vesicles (OMVs), they wondered if immunization with outer membrane vesicles (OMVs) would also elicit lung T-cell response. Similar to mucosal immunization with heat-killed K. pneumoniae, mucosal immunization with OMVs derived from K2 K. pneumoniae elicit robust lung Th17 and Th1 responses. These OMVs also elicit local IgG and IgA response. To determine which outer membrane proteins are packaged into these OMVs, the inventors conducted tandem mass spectroscopy on these and detected several OMPs including OmpA, Omp36k, OmpLolb, OmpW, OmpX, and OmpC. These proteins have been expressed in E. coli. As discussed above, when OMPx was formulated with LPS as a pre-clinical TLR4 adjuvant induced lung Th17 responses without affecting lung γδT-cell populations. Heterologous challenge with 10³ of KP396—a mucoid antibiotic-resistant strain showed robust growth in control immunized mice but minimal growth after OmpX immunization. To assess vaccine efficacy, mice were again challenged with 10³ of KP396 intranasally three-weeks post-immunization. OmpX alone was ineffective in bacterial control but when adjuvanted with LTA1 or dmLT showed improved bacterial control (n=5-7 per group, p<0.05, ANOVA, Kruskal-Wallis, with correction for multiple comparisons). In the intranasal immunization arm, Ompx formulated with dmLT or LTA1 has similar efficacy of OMV mucosal immunization.

Thus, although OmpX looks like an excellent vaccine candidate, it is a single antigen and thus resistance could evolve. Thus, the inventors investigated multi-valent Omp based vaccines. They prioritized Omps for several reasons. First, they can be produced in E. coli. Second, they have the ability to elicit robust T-cell and B-cell responses. And third, their conservation across many related MDR ESKPAE pathogens makes broad-spectrum efficacy possible. Based on the fact that they were present in outer membrane vesicles, they show a very high degree of homology to other Omps in Enterobacteriaceae family. For example, OmpX from K. pneumoniae has 98.6% homogly with K. oxytoca, Enterobacter cloacae and 98.6% with E. coli. OmpC has 94.5% homogly with K. oxytoca, 89.3% Enterobacter cloacae and 95.1% with E. coli. OmpW has 99.1% homogly with K. oxytoca, 94.3% Enterobacter cloacae and 99.1% with E. coli. OmpLolB has 99.5% homogly with K. oxytoca, 91.4% Enterobacter cloacae and 99.5% with E. coli. Thus, one would predict cross reactive T cell and B cell immunity to these antigens.

These and other aspects of the disclosure are set out in detail below.

### I. BACTERIAL INFECTIONS AND RESISTANCE

A. Infections and Resistant Infections

Bacteria are ubiquitous in virtually any environment, including hose thought of as sterile, and in many aspects they are not only beneficial but essential to survival of all life. However, bacteria are also a major health threat in both industrialized and third world countries. Moreover, the growing ability for bacteria to evade current antimicrobial therapies is a major health risk for the entire world.

The classic symptoms of a bacterial infection are localized redness, heat, swelling and pain. One of the hallmarks of a bacterial infection is local pain, pain that is in a specific part of the body. For example, if a cut occurs and is infected with bacteria, pain occurs at the site of the infection. Bacterial throat pain is often characterized by more pain on one side of the throat. An ear infection is more likely to be diagnosed as bacterial if the pain occurs in only one ear. A cut that produces pus and milky-colored liquid is most likely infected.

There is a general chain of events that applies to infections. The chain of events involves several steps, which include the infectious agent, reservoir, entering a susceptible host, exit and transmission to new hosts. Each of the links must be present in a chronological order for an infection to develop.

Infection begins when an organism successfully enters the body, grows and multiplies. This is referred to as colonization. Most humans are not easily infected. Those who are weak, sick, malnourished, have cancer or are diabetic have increased susceptibility to chronic or persistent infections. Individuals who have a suppressed immune system are particularly susceptible to opportunistic infections. Entrance to the host at host-pathogen interface, generally occurs through the mucosa in orifices like the oral cavity, nose, eyes, genitalia, anus, or the microbe can enter through open wounds. While a few organisms can grow at the initial site of entry, many migrate and cause systemic infection in different organs. Some pathogens grow within the host cells (intracellular) whereas others grow freely in bodily fluids.

Wound colonization refers to nonreplicating microorganisms within the wound, while in infected wounds, replicating organisms exist and tissue is injured. All multicellular organisms are colonized to some degree by extrinsic organisms, and the vast majority of these exist in either a mutualistic or commensal relationship with the host. An example of the former is the anaerobic bacteria species, which colonizes the mammalian colon, and an example of the latter are the various species of Staphylococcus that exist on human skin. Neither of these colonizations are considered infections. The difference between an infection and a colonization is often only a matter of circumstance. Non-pathogenic organisms can become pathogenic given specific conditions, and even the most virulent organism requires certain circumstances to cause a compromising infection. Some colonizing bacteria, such as Corynebacteria sp. and Viridans streptococci, prevent the adhesion and colonization of pathogenic bacteria and thus have a symbiotic relationship with the host, preventing infection and speeding wound healing.

The variables involved in the outcome of a host becoming inoculated by a pathogen and the ultimate outcome include:
the route of entry of the pathogen and the access to host regions that it gains
the intrinsic virulence of the particular organism
the quantity or load of the initial inoculant
the immune status of the host being colonized
   As an example, several staphylococcal species remain harmless on the skin, but, when present in a normally sterile space, such as in the capsule of a joint or the peritoneum, multiply without resistance and cause harm.

Disease can arise if the host's protective immune mechanisms are compromised and the organism inflicts damage on the host. Microorganisms can cause tissue damage by releasing a variety of toxins or destructive enzymes. For example, Clostridium tetani releases a toxin that paralyzes muscles, and Staphylococcus releases toxins that produce shock and sepsis. Persistent infections occur because the body is unable to clear the organism after the initial infection. Persistent infections are characterized by the continual presence of the infectious organism, often as latent infection with occasional recurrent relapses of active infection.

Antimicrobial resistance (AMR or AR) is the ability of a microbe to resist the effects of medication that once could successfully treat the microbe. The term antibiotic resistance (AR or ABR) is a subset of AMR, as it applies only to bacteria becoming resistant to antibiotics. Resistant microbes are more difficult to treat, requiring alternative medications or higher doses of antimicrobials. These approaches may be more expensive, more toxic or both. Microbes resistant to multiple antimicrobials are called multidrug resistant (MDR). Those considered extensively drug resistant (XDR) or totally drug resistant (TDR) are sometimes called “superbugs”.

Resistance arises through one of three mechanisms: natural resistance in certain types of bacteria, genetic mutation, or by one species acquiring resistance from another. Resistance can appear spontaneously because of random mutations. However, extended use of antimicrobials appears to encourage selection for mutations which can render antimicrobials ineffective.

Preventive measures include only using antibiotics when needed, thereby stopping misuse of antibiotics. Narrow-spectrum antibiotics are preferred over broad-spectrum antibiotics when possible, as effectively and accurately targeting specific organisms is less likely to cause resistance, as well as side effects. For people who take these medications at home, education about proper use is essential. Health care providers can minimize spread of resistant infections by use of proper sanitation and hygiene, including handwashing and disinfecting between patients, and should encourage the same of the patient, visitors, and family members.

Rising drug resistance is caused mainly by use of antimicrobials in humans and other animals and spread of resistant strains between the two. Growing resistance has also been linked to dumping of inadequately treated effluents from the pharmaceutical industry, especially in countries where bulk drugs are manufactured. Antibiotics increase selective pressure in bacterial populations, causing vulnerable bacteria to die; this increases the percentage of resistant bacteria which continue growing. Even at very low levels of antibiotic, resistant bacteria can have a growth advantage and grow faster than vulnerable bacteria. With resistance to antibiotics becoming more common there is greater need for alternative treatments. Calls for new antibiotic therapies have been issued, but new drug development is becoming rarer.

Antimicrobial resistance is increasing globally because of greater access to antibiotic drugs in developing countries. Estimates are that 700,000 to several million deaths result per year. Each year in the United States, at least 2 million people become infected with bacteria that are resistant to antibiotics and at least 23,000 people die as a result. There are public calls for global collective action to address the threat that include proposals for international treaties on antimicrobial resistance. Worldwide antibiotic resistance is not completely identified, but poorer countries with weaker healthcare systems are more affected.

B. Bovine Mastitis

Bovine mastitis is the persistent, inflammatory reaction of the udder tissue due to physical trauma or microorganisms infections. Mastitis, a potentially fatal mammary gland infection, is the most common disease in dairy cattle in the United States and worldwide. It is also the most costly disease to the dairy industry. Milk from cows suffering from mastitis have an increased somatic cell count.

Mastitis occurs when white blood cells (leukocytes) are released into the mammary gland, usually in response to bacteria invading the teat canal or occasionally by chemical, mechanical, or thermal trauma on the udder. Milk-secreting tissue and various ducts throughout the mammary gland are damaged due to toxins released by the bacteria resulting in reduced milk yield and quality.

This disease can be identified by abnormalities in the udder such as swelling, heat, redness, hardness, or pain (if it is clinical). Other indications of mastitis may be abnormalities in milk such as a watery appearance, flakes, or clots. When infected with sub-clinical mastitis, a cow does not show any visible signs of infection or abnormalities in milk or on the udder.

Bacteria that are known to cause mastitis include Pseudomonas aeruginosa, Staphylococcus aureus, Staphylococcus epidermidis, Streptococcus agalactiae, Streptococcus uberis, Brucella melitensis, Corynebacterium bovis, Mycoplasma spp. (including Mycoplasma bovis), Escherichia coli (E. coli), Klebsiella pneumoniae, Klebsiella oxytoca, Enterobacter aerogenes, Pasteurella spp., Trueperella pyogenes^{[5]}(previously Arcanobacterium pyogenes), Proteus spp., Prototheca zopfii (achlorophyllic algae) and Prototheca wickerhamii (achlorophyllic algae). These bacteria can be classified as environmental or contagious depending on mode and source of transmission.

Mastitis is most often transmitted by repetitive contact with the milking machine, and through contaminated hands or materials. Another route is via the oral-to-udder transmission among calves. Feeding calves on milk may introduce some mastitis causing bacteria strain in the oral cavity of the calf where it will stay dormant until it is transmitted elsewhere. Since grouped calves like to stimulate suckling, they will transmit the bacteria to the udder tissue of their fellow calves. The bacteria will lay dormant in the udder tissue as the calf grows until it begins to lactate. That is when the bacteria activates and causes mastitis. This calls for strict calf management practices to curb this route of transmission.

Cattle affected by mastitis can be detected by examining the udder for inflammation and swelling, or by observing the consistency of the milk, which will often develop clots or change color when a cow is infected. Another method of detection is the California mastitis test, which is designed to measure the milk's somatic cell count as a means for detecting inflammation and infection of the udder.

Treatment is possible with long-acting antibiotics, but milk from such cows is not marketable until drug residues have left the cow's system. Antibiotics may be systemic (injected into the body), or they may be forced upwards into the teat through the teat canal (intramammary infusion). Cows being treated may be marked with tape to alert dairy workers, and their milk is syphoned off and discarded. To determine whether the levels of antibiotic residuals are within regulatory requirements, special tests exist. Vaccinations for mastitis are available, but as they only reduce the severity of the condition, and cannot prevent reoccurring infections, they should be used in conjunction with a mastitis prevention program.

Practices such as good nutrition, proper milking hygiene, and the culling of chronically infected cows can help. Ensuring that cows have clean, dry bedding decreases the risk of infection and transmission. Dairy workers should wear rubber gloves while milking, and machines should be cleaned regularly to decrease the incidence of transmission. A good milking routine is vital. This usually consists of applying a pre-milking teat dip or spray, such as an iodine spray, and wiping teats dry prior to milking. The milking machine is then applied. After milking, the teats can be cleaned again to remove any growth medium for bacteria. A post milking product such as iodine-propylene glycol dip is used as a disinfectant and a barrier between the open teat and the bacteria in the air. Mastitis can occur after milking because the teat holes close after 15 minutes if the animal sits in a dirty place with feces and urine.

### II. KLEBSIELLA AND STRUCTURALLY RELATED ORGANISMS

A. Klebsiella

Klebsiella is a genus of nonmotile, Gram-negative, oxidase-negative, rod-shaped bacteria with a prominent polysaccharide-based capsule. Klebsiella species are found everywhere in nature. This is thought to be due to distinct sublineages developing specific niche adaptations, with associated biochemical adaptations which make them better suited to a particular environment. They can be found in water, soil, plants, insects and other animals including humans.

The members of the genus Klebsiella are a part of the human and animal's normal flora in the nose, mouth and intestines. The species of Klebsiella are all Gram-negative and nonmotile. They tend to be shorter and thicker when compared to others in the Enterobacteriaceae family. The cells are rods in shape and generally measures 0.3 to 1.5 μm wide by 0.5 to 5.0 μm long. They can be found singly, in pairs, in chains or linked end to end. Klebsiella can grow on ordinary lab medium and do not have special growth requirements, like the other members of Enterobacteriaceae. The species are aerobic but facultatively anaerobic. Their ideal growth temperature is 35° to 37° C., while their ideal pH level is about 7.2. Examples include:

K. granulomatis
K. oxytoca
K. michiganensis
K. pneumoniae (type-species)
K. p. subsp. ozaenae
K. p. subsp. pneumoniae
K. p. subsp. rhinoscleromatis
K. quasipneumoniae
K. q. subsp. quasipneumoniae
K. q. subsp. similipneumoniae
K. grimontii
K. variicola

Klebsiella bacteria tend to be rounder and thicker than other members of the Enterobacteriaceae family. They typically occur as straight rods with rounded or slightly pointed ends. They can be found singly, in pairs, or in short chains. Diplobacillary forms are commonly found in vivo. They have no specific growth requirements and grow well on standard laboratory media but grow best between 35 and 37° C. and at pH 7.2. The species are facultative anaerobes, and most strains can survive with citrate and glucose as their sole carbon sources and ammonia as their sole nitrogen source.

Members of the genus produce a prominent capsule, or slime layer, which can be used for serologic identification, but molecular serotyping may replace this method. Members of the genus Klebsiella typically express two types of antigens on their cell surfaces. The first, 0 antigen, is a component of the lipopolysaccharide (LPS), of which 9 varieties exist. The second is K antigen, a capsular polysaccharide with more than 80 varieties. Both contribute to pathogenicity and form the basis for serogrouping.

Klebsiella species are routinely found in the human nose, mouth, and gastrointestinal tract as normal flora; however, they can also behave as opportunistic human pathogens. Klebsiella species are known to also infect a variety of other animals, both as normal flora and opportunistic pathogens.

Klebsiella organisms can lead to a wide range of disease states, notably pneumonia, urinary tract infections, septicemia, meningitis, diarrhea, and soft tissue infections. Klebsiella species have also been implicated in the pathogenesis of ankylosing spondylitis and other spondyloarthropathies. The majority of human Klebsiella infections are caused by K. pneumoniae, followed by K. oxytoca. Infections are more common in the very young, very old, and those with other underlying diseases, such as cancer, and most infections involve contamination of an invasive medical device.

During the last 40 years, many trials for constructing effective K. pneumoniae vaccines have been tried. Currently, no Klebsiella vaccine has been licensed for use in the U.S. K. pneumoniae is the most common cause of nosocomial respiratory tract and premature intensive care infections, and the second-most frequent cause of Gram-negative bacteremia and urinary tract infections. Drug-resistant isolates remain an important hospital-acquired bacterial pathogen, add significantly to hospital stays, and are especially problematic in high-impact medical areas such as intensive care units. This antimicrobial resistance is thought to be attributable mainly to multidrug efflux pumps. The ability of K. pneumoniae to colonize the hospital environment, including carpeting, sinks, flowers, and various surfaces, as well as the skin of patients and hospital staff, has been identified as a major factor in the spread of hospital-acquired infections.

In addition to certain Klebsiella spp. being discovered as human pathogens, others such as K. variicola have been identified as emerging pathogens in humans and animals alike. For instance, K. variicola has been identified as one of the causes of bovine mastitis. In plant systems, Klebsiella can be found in a variety of plant hosts. K. pneumoniae and K. oxytoca are able to fix atmospheric nitrogen into a form that can be used by plants, thus are called associative nitrogen fixers or diazotrophs. The bacteria attach strongly to root hairs and less strongly to the surface of the zone of elongation and the root cap mucilage. They are bacteria of interest in an agricultural context, due to their ability to increase crop yields under agricultural conditions. Their high numbers in plants are thought to be at least partly attributable to their lack of a flagellum, as flagella are known to induce plant defenses. Additionally, K. variicola is known to associate with a number of different plants including banana trees, sugarcane and has been isolated from the fungal gardens of leaf-cutter ants.

B. Other Organisms

1. E. coli

Escherichia coli is a Gram-negative, facultative anaerobic, rod-shaped, coliform bacterium of the genus Escherichia that is commonly found in the lower intestine of warm-blooded organisms (endotherms). Most E. coli strains are harmless, but some serotypes can cause serious food poisoning in their hosts and are occasionally responsible for product recalls due to food contamination. The harmless strains are part of the normal microbiota of the gut, and can benefit their hosts by producing vitamin K2, and preventing colonization of the intestine with pathogenic bacteria, having a symbiotic relationship. E. coli is expelled into the environment within fecal matter. The bacterium grows massively in fresh fecal matter under aerobic conditions for 3 days, but its numbers decline slowly afterwards.

E. coli and other facultative anaerobes constitute about 0.1% of gut microbiota, and fecal-oral transmission is the major route through which pathogenic strains of the bacterium cause disease. Cells are able to survive outside the body for a limited amount of time, which makes them potential indicator organisms to test environmental samples for fecal contamination. A growing body of research, though, has examined environmentally persistent E. coli which can survive for extended periods outside a host.

The bacterium can be grown and cultured easily and inexpensively in a laboratory setting and has been intensively investigated for over 60 years. E. coli is a chemoheterotroph whose chemically defined medium must include a source of carbon and energy. E. coli is the most widely studied prokaryotic model organism, and an important species in the fields of biotechnology and microbiology, where it has served as the host organism for the majority of work with recombinant DNA. Under favorable conditions, it takes up to 20 minutes to reproduce.

E. coli is a Gram-negative, facultative anaerobic (that makes ATP by aerobic respiration if oxygen is present but is capable of switching to fermentation or anaerobic respiration if oxygen is absent) and non-sporulating bacterium. Cells are typically rod-shaped and are about 2.0 μm long and 0.25-1.0 μm in diameter, with a cell volume of 0.6-0.7 μm³.

E. coli stains Gram-negative because its cell wall is composed of a thin peptidoglycan layer and an outer membrane. During the staining process, E. coli picks up the color of the counterstain safranin and stains pink. The outer membrane surrounding the cell wall provides a barrier to certain antibiotics such that E. coli is not damaged by penicillin.

Strains that possess flagella are motile. The flagella have a peritrichous arrangement. It also attaches and effaces to the microvilli of the intestines via an adhesion molecule known as intimin.

E. coli can live on a wide variety of substrates and uses mixed-acid fermentation in anaerobic conditions, producing lactate, succinate, ethanol, acetate, and carbon dioxide. Since many pathways in mixed-acid fermentation produce hydrogen gas, these pathways require the levels of hydrogen to be low, as is the case when E. coli lives together with hydrogen-consuming organisms, such as methanogens or sulphate-reducing bacteria.

E. coli and related bacteria possess the ability to transfer DNA via bacterial conjugation or transduction, which allows genetic material to spread horizontally through an existing population. The process of transduction, which uses the bacterial virus called a bacteriophage, is where the spread of the gene encoding for the Shiga toxin from the Shigella bacteria to E. coli helped produce E. coli O157:H7, the Shiga toxin-producing strain of E. coli.

E. coli belongs to a group of bacteria informally known as coliforms that are found in the gastrointestinal tract of warm-blooded animals. E. coli normally colonizes an infant's gastrointestinal tract within 40 hours of birth, arriving with food or water or from the individuals handling the child. In the bowel, E. coli adheres to the mucus of the large intestine. It is the primary facultative anaerobe of the human gastrointestinal tract. (Facultative anaerobes are organisms that can grow in either the presence or absence of oxygen.) As long as these bacteria do not acquire genetic elements encoding for virulence factors, they remain benign commensals.

Most E. coli strains do not cause disease, but virulent strains can cause gastroenteritis, urinary tract infections, neonatal meningitis, hemorrhagic colitis, and Crohn's disease. Common signs and symptoms include severe abdominal cramps, diarrhea, hemorrhagic colitis, vomiting, and sometimes fever. In rarer cases, virulent strains are also responsible for bowel necrosis (tissue death) and perforation without progressing to hemolytic-uremic syndrome, peritonitis, mastitis, septicemia, and Gram-negative pneumonia. Very young children are more susceptible to develop severe illness, such as hemolytic uremic syndrome, however, healthy individuals of all ages are at risk to the severe consequences that may arise as a result of being infected with E. coli.

Some strains of E. coli for example O157:H7, can produce Shiga toxin (classified as a bioterrorism agent). This toxin causes premature destruction of the red blood cells, which then clog the body's filtering system, the kidneys, causing hemolytic-uremic syndrome (HUS). Unlike most E. coli that naturally live in the gut, the Shiga toxin that causes inflammatory responses in target cells of the gut (the lesions the toxin leaves behind are the reason why bloody diarrhea is a symptom of an Shiga toxin producing E. coli infection). [In some rare cases (usually in children and the elderly) Shiga toxin producing E. coli infection may lead to hemolytic uremic syndrome (HUS), which can cause kidney failure and even death. Signs of hemolytic uremic syndrome, include decreased frequency of urination, lethargy, and paleness of cheeks and inside the lower eyelids. In 25% of HUS patients, complications of nervous system occur, which in turn causes strokes due to small clots of blood which lodge in capillaries in the brain. This causes the body parts controlled by this region of the brain not to work properly. In addition, this strain causes the buildup of fluid (since the kidneys do not work), leading to edema around the lungs and legs and arms. This increase in fluid buildup especially around the lungs impedes the functioning of the heart, causing an increase in blood pressure.

Uropathogenic E. coli (UPEC) is one of the main causes of urinary tract infections. It is part of the normal microbiota in the gut and can be introduced in many ways. In particular for females, the direction of wiping after defecation (wiping back to front) can lead to fecal contamination of the urogenital orifices. Anal intercourse can also introduce this bacterium into the male urethra, and in switching from anal to vaginal intercourse, the male can also introduce UPEC to the female urogenital system. For more information, see the databases at the end of the article or UPEC pathogenicity.

2. E. cloacae

Enterobacter cloacae is a clinically significant Gram-negative, facultatively-anaerobic, rod-shaped bacterium. In microbiology labs, E. cloacae is frequently grown at 30° C. on nutrient agar or broth or at 35° C. in tryptic soy broth. It is a rod-shaped, Gram-negative bacterium, is facultatively anaerobic, and bears peritrichous flagella. It is oxidase-negative and catalase-positive.

Enterobacter cloacae has been used in a bioreactor-based method for the biodegradation of explosives and in the biological control of plant diseases. E. cloacae is considered a biosafety level 1 organism in the United States and level 2 in Canada. Enterobacter cloacae is a member of the normal gut flora of many humans and is not usually a primary pathogen. Some strains have been associated with urinary tract and respiratory tract infections in immunocompromised individuals. Treatment with cefepime and gentamicin has been reported.

E. cloacae was described for the first time in 1890 as Bacillus cloacae, and then underwent numerous taxonomical changes, becoming ‘Bacterium cloacae’ in 1896, Cloaca cloacae in 1919, it was identified as ‘Aerobacter cloacae’ in 1923, Aerobacter cloacae in 1958 and E. cloacae in 1960, by which it is still known today. E. cloacae is ubiquitous in terrestrial and aquatic environments (water, sewage, soil and food). These strains occur as commensal microflora in the intestinal tracts of humans and animals and play an important role as pathogens in plants and insects. This diversity of habitats is mirrored by the genetic variety of the nomenspecies E. cloacae. E. cloacae is also an important nosocomial pathogen responsible for bacteremia and lower respiratory tract, urinary tract and intra-abdominal infections, as well as endocarditis, septic arthritis, osteomyelitis and skin and soft tissue infections. The skin and the GI tract are the most common sites through which E. cloacae can be contracted.

E. cloacae tends to contaminate various medical, intravenous and other hospital devices. Nosocomial outbreaks have also been associated with colonization of certain surgical equipment and operative cleaning solutions. Another potential reservoir for nosocomial bacteremia is the heparin solution used to irrigate certain intravascular devices continually. This fluid had been implicated as a reservoir for outbreaks of device-associated bacteremia in several instances.

In recent years, E. cloacae has emerged as one of the most commonly found nosocomial pathogen in neonatal units, with several outbreaks of infection being reported. In 1998, an outbreak in a neonatal intensive care unit resulted in nine deaths, and in 2003, three outbreaks with 42 systemic infections and a mortality of 34% occurred. This microorganism may be transmitted to neonates through contaminated intravenous fluids, total parenteral nutrition solutions and medical equipment. Many single-clone outbreaks, probably caused by cross-transmission via healthcare workers, have been described, suggesting that inpatients can also act as a reservoir. The type strains of the species are E. cloacae ATCC 49162 and 13047. This latter strain is the first complete genome sequence of the E. cloacae species and the type strain is E. cloacae subsp. cloacae.

The complete E. cloacae subsp. cloacae ATCC 13047 genome contains a single circular chromosome of 5,314,588 bp and two circular plasmids, pECL_A and pECL_B, of 200,370 and 85,650 bp (GenBank accession numbers CP001918, CP001919 and CP001920, respectively). Other genomes of E. cloacae that have been sequenced are deposited in GenBank under accession numbers CP002272, CP002886, FP929040 and AGSY00000000.

### III. THERAPIES

In some aspects of the present disclosure, the vaccines disclosed herein may be used to treat a bacterial infection. While humans contain numerous different bacteria on and inside their bodies, an imbalance in bacterial levels or the introduction of pathogenic bacteria can cause a symptomatic bacterial infection. Additionally, different bacteria have a wide range of interactions with body and those interactions can modulate ability of the bacteria to cause an infection. For example, bacteria can be conditionally pathogenic such that they only cause an infection under specific conditions. For example, bacteria exist in the normal human bacterial biome, but these bacteria when they are allowed to colonize other parts of the body causing a skin infection, pneumonia, or sepsis. Other bacteria are known as opportunistic pathogens and only cause diseases in a patient with a weakened immune system or another disease or disorder.

Bacterial infections could be targeted to a specific location in or on the body. For example, bacteria could be harmless if only exposed to the specific organs, but when it comes in contact with a specific organ or tissue, the bacteria can begin replicating and cause a bacterial infection.

In particular, the inventors contemplate treatment of bacterial infections, including those caused by Klebsiella, and structurally-related pathogens such as E. coli and E. cloacae. These organisms have a remarkable ability to accumulate additional antibiotic resistance determinants, resulting in the formation of multiply-drug-resistant strains.

A. Vaccine Components

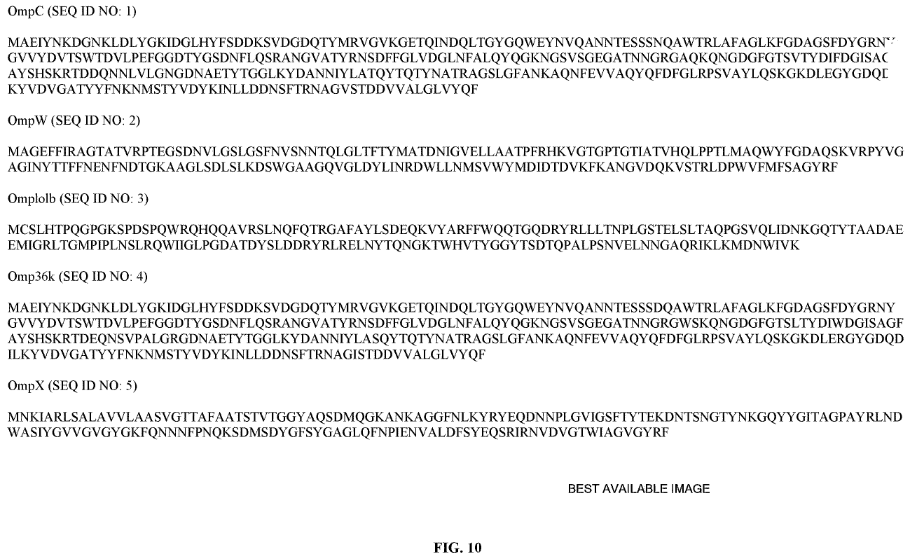

The inventors contemplate employing multivalent vaccines including three or more of the following outer membrane protesin (OMPs) from Klebsiella: OmpC, OmpW, Omplolb and Omp36K. The sequences for these proteins are provided in FIG. 10. Thus, the possible combinations include:
OmpC, OmpW, Omplolb and Omp36K
OmpC, OmpW, and Omplolb
OmpC, OmpW, and Omp36K
OmpC, Omplolb and Omp36K
OmpW, Omplolb and Omp36K

B. Pharmaceutical Formulations and Routes of Administration Where clinical applications are contemplated, it will be necessary to prepare pharmaceutical compositions in a form appropriate for the intended application. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Buffers also will be employed when recombinant cells are introduced into a patient. Aqueous compositions of the present disclosure comprise an effective amount of the vector to cells, dissolved or dispersed in a pharmaceutically acceptable carrier or aqueous medium. Such compositions also are referred to as inocula. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the vectors or cells of the present disclosure, its use in therapeutic compositions is contemplated. Supplementary active ingredients also can be incorporated into the compositions.

The active compositions of the present disclosure may include classic pharmaceutical preparations. Administration of these compositions according to the present disclosure will be via any common route so long as the target tissue is available via that route. Such routes include oral, nasal, buccal, rectal, vaginal or topical route. Alternatively, administration may be by orthotopic, intradermal, subcutaneous, intramuscular, intranasal, intraperitoneal, or intravenous injection. Such compositions would normally be administered as pharmaceutically acceptable compositions, described supra.

The active compounds may also be administered parenterally or intraperitoneally. Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

For oral administration the uncialamycin derivatives of the present disclosure may be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). Alternatively, the active ingredient may be incorporated into an antiseptic wash containing sodium borate, glycerin and potassium bicarbonate. The active ingredient may also be dispersed in dentifrices, including: gels, pastes, powders and slurries. The active ingredient may be added in a therapeutically effective amount to a paste dentifrice that may include water, binders, abrasives, flavoring agents, foaming agents, and humectants.

The compositions of the present disclosure may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences,” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

C. Adjuvants

As is well known in the art, the immunogenicity of a particular immunogen composition can be enhanced by the use of non-specific stimulators of the immune response, known as adjuvants. Adjuvants have been used experimentally to promote a generalized increase in immunity against poorly immunogenic antigens. Immunization protocols have used adjuvants to stimulate responses for many years, and as such adjuvants are well known to one of ordinary skill in the art. Some adjuvants affect the way in which antigens are presented. For example, the immune response is increased when protein antigens are adsorbed to alum. Emulsification of antigens also prolongs the duration of antigen presentation and initiates an innate immune response. Suitable molecule adjuvants include all acceptable immunostimulatory compounds, such as cytokines, toxins or synthetic compositions.

In some aspects, the compositions described herein may further comprise another adjuvant. Although alum is an approved adjuvant for humans, adjuvants in experimental animals include complete Freund's adjuvant (a non-specific stimulator of the immune response containing killed Mycobacterium tuberculosis), incomplete Freund's adjuvants and aluminum hydroxide adjuvant. Other adjuvants that may also be used in animals and sometimes humans include Interleukin (IL)-1, IL-2, IL-4, IL-7, IL-12, interferon, Bacillus Calmette-Guérin (BCG), aluminum hydroxide, muramyl dipeptide (MDP) compounds, such as thur-MDP and nor-MDP (N-acetylmuramyl-L-alanyl-D-isoglutamine MDP), lipid A, and monophosphoryl lipid A (MPL). RIBI, which contains three components extracted from bacteria, MPL, trehalose dimycolate (TDM) and cell wall skeleton (CWS) in a 2% squalene/Tween 80 emulsion also is contemplated. MEW antigens may even be used.

Some adjuvants, for example, certain organic molecules obtained from bacteria, act on the host rather than on the antigen. An example is MDP, a bacterial peptidoglycan. The effects of MDP, as with most adjuvants, are not fully understood, although researchers are now beginning to understand that they activate cells of the innate immune system, e.g. dendritic cells, macrophages, neutrophils, NKT cells, NK cells, etc. MDP stimulates macrophages but also appears to stimulate B cells directly. The effects of adjuvants, therefore, are not antigen-specific. If they are administered together with a purified antigen, however, they can be used to selectively promote the response to the antigen.

Various polysaccharide adjuvants may also be used. For example, the use of various pneumococcal polysaccharide adjuvants on the antibody responses of mice has been described (Yin et al., 1989). The doses that produce optimal responses, or that otherwise do not produce suppression, should be employed as indicated (Yin et al., 1989). Polyamine varieties of polysaccharides are particularly contemplated, such as chitin and chitosan, including deacetylated chitin.

Another group of adjuvants are the muramyl dipeptide (MDP, N-acetylmuramyl-L-alanyl-D-isoglutamine) group of bacterial peptidoglycans. Derivatives of muramyl dipeptide, such as the amino acid derivative threonyl-MDP, and the fatty acid derivative muramyl tripeptide phosphatidylethanolamine (MTPPE) are also contemplated.

BCG and BCG-cell wall skeleton (CWS) may also be used as adjuvants, with or without trehalose dimycolate. Trehalose dimycolate may be used itself. Trehalose dimycolate administration has been shown to correlate with augmented resistance to influenza virus infection in mice (Azuma et al., 1988). Trehalose dimycolate may be prepared as described in U.S. Pat. No. 4,579,945. BCG is an important clinical tool because of its immunostimulatory properties. BCG acts to stimulate the reticuloendothelial system (RES), activates natural killer (NK) cells and increases proliferation of hematopoietic stem cells. Cell wall extracts of BCG have proven to have excellent immune adjuvant activity. Molecular genetic tools and methods for mycobacteria have provided the means to introduce foreign genes into BCG (Jacobs et al., 1987; Snapper et al., 1988; Husson et al., 1990; Martin et al., 1990). Live BCG is an effective and safe vaccine used worldwide to prevent tuberculosis. BCG and other mycobacteria are highly effective adjuvants, and the immune response to mycobacteria has been studied extensively. With nearly 2 billion immunizations, BCG has a long record of safe use in man (Luelmo, 1982; Lotte et al., 1984). It is one of the few vaccines that can be given at birth, it engenders long-lived immune responses with only a single dose, and there is a worldwide distribution network with experience in BCG vaccination. An exemplary BCG vaccine is sold as TICE BCG (Organon Inc., West Orange, N.J.).

Amphipathic and surface-active agents, e.g., saponin and derivatives such as QS21 (Cambridge Biotech), form yet another group of adjuvants for use with the immunogens of the present disclosure. Nonionic block copolymer surfactants (Rabinovich et al., 1994) may also be employed. Oligonucleotides are another useful group of adjuvants (Yamamoto et al., 1988). Quil A and lentinen are other adjuvants that may be used in certain embodiments of the present disclosure.

Another group of adjuvants are the detoxified endotoxins, such as the refined detoxified endotoxin of U.S. Pat. No. 4,866,034. These refined detoxified endotoxins are effective in producing adjuvant responses in mammals. Of course, the detoxified endotoxins may be combined with other adjuvants to prepare multi-adjuvant-incorporated cells. For example, combination of detoxified endotoxins with trehalose dimycolate is particularly contemplated, as described in U.S. Pat. No. 4,435,386. Combinations of detoxified endotoxins with trehalose dimycolate and endotoxic glycolipids is also contemplated (U.S. Pat. No. 4,505,899), as is combination of detoxified endotoxins with cCWS or CWS and trehalose dimycolate, as described in U.S. Pat. Nos. 4,436,727, 4,436,728 and 4,505,900. Combinations of just CWS and trehalose dimycolate, without detoxified endotoxins, are also envisioned to be useful, as described in U.S. Pat. No. 4,520,019.

Those of skill in the art will know the different kinds of adjuvants that can be conjugated to vaccines in accordance with this disclosure and which are approved for human vs experimental use. These include alkyl lysophospholipids (ALP); BCG; and biotin (including biotinylated derivatives) among others. Certain adjuvants particularly contemplated for use are the teichoic acids from Gram⁻ bacterial cells. These include the lipoteichoic acids (LTA), ribitol teichoic acids (RTA) and glycerol teichoic acid (GTA). Active forms of their synthetic counterparts may also be employed in connection with the compositions of this disclosure (Takada et al., 1995).

Of particular interest in accordance with the present disclosure is the adjuvant LTA1. LTA1 is an adjuvant based on the A-subunit of the heat-labile enterotoxin from Escherichia coli (LT). LTA1 is a 21 kDA protein that contains the active domain of LT toxin and can ADP-ribosylate other proteins initiating activation of antigen-presenting cells (APCs). LTA1 treatment of APCs results in protein kinase A and inflammasome activation leading to potent cytokine secretion, including IL-1beta. LTA1 admixed with vaccine antigens and administered intranasally or by intrapulmonary delivery, promotes antigen-specific memory responses. Importantly, LTA1 is not known to alter any neurological functions, unlike LT or cholera toxin. This is of note, as the enterotoxin family of adjuvants are powerful mucosal adjuvants but have been hindered by major safety concerns in past clinical trials, particularly for intranasal delivery. LTA1 is a safe and effective mucosal adjuvant because it can achieve broad mucosal and systemic immunity without the potential safety risks of the parent proteins and high levels of immunogenicity (i.e., anti-LT antibodies).

Another adjuvant if particular interest here is dmLT. dmLT, or more technically LT(R192G/L211A), is an 84-kDa polymeric protein with an AB5 structure composed of an enzymatically active A subunit (28 kDa) noncovalently associated with a pentameric B subunit (consisting of five 11.5-kDa monomers). dmLT is distinguished from its parent molecule heat-labile enterotoxin (LT) by the substitution of two residues in the A subunit, a glycine for an arginine at amino acid 192 (R192G) and an alanine for a leucine at amino acid 211 (L211A). dmLT enhances vaccine-specific systemic and mucosal immune responses following mucosal or parenteral delivery.

Like LTA1, dmLT promotes immunity to antigens that are codelivered after simply admixing dmLT and the antigen in aqueous buffer. Thus, unlike many depot-type adjuvants, such as aluminum hydroxide, no advanced preparation or absorption is required to formulate the antigen/adjuvant vaccine. dmLT can be formulated with the antigen at either the point of manufacture or the point of delivery.

Through the combined action of dmLT's immunostimulatory properties and universal cell binding, uptake of codelivered antigens is enhanced and mucosal immunity is promoted. This enables the delivery of immunization formulations (most strikingly for subunit vaccines) at previously inaccessible sites, such as in oral (p.o.), sublingual (s.l.), transcutaneous (t.c.i.), etc., delivery. Many of these approaches are needle free and have the potential to increase ease of administration and compliance and lower the risk of disease outbreaks from unsafe injections.

Unlike other adjuvants, such as aluminum hydroxide or many Toll-like receptor (TLR)-based adjuvants (e.g., monophosphoryl lipid A (MPL) and CpG), dmLT and LTA1 induce strong interleukin-17 (IL-17) recall cytokine secretion and antigen-specific Th17 responses after parenteral or mucosal immunization. This is a newly appreciated arm of the adaptive immune response that is critical in protection from pathogens, particularly in preventing infections in mucosal tissue and control of bacterial infections. In addition, IL-17 secretion enhances the availability of mucosal antibodies by upregulating polymeric Ig receptor levels in epithelial cells, increasing transport of secretory IgA (sIgA) into the lumen of mucosal tissue, and promoting T-independent B-cell differentiation into IgA-secreting cells.

Last, dmLT promotes the development of mucosal immune responses following parenteral immunization. While these observations are validated only in preclinical animal models thus far, this is a distinction from most vaccines delivered by parenteral injection, which can induce serum antibodies and cell-mediated immunity but only limited or nonexistent responses at mucosal surfaces.

When considering the vaccine options listed above, the vaccines may include the following adjuvanted possibilities:
OmpC, OmpW, Omplolb and Omp36K+LTAI
OmpC, OmpW, Omplolb and Omp36K+dmLT
OmpC, OmpW, Omplolb and Omp36K+LTAI+dmLT
OmpC, OmpW, and Omplolb+LTAI
OmpC, OmpW, and Omplolb+dmLT
OmpC, OmpW, and Omplolb+LTAI+dmLT
OmpC, OmpW, and Omp36K+LTAI
OmpC, OmpW, and Omp36K+dmLT
OmpC, OmpW, and Omp36K+LTAI+dmLT
OmpC, Omplolb and Omp36K+LTAI
OmpC, Omplolb and Omp36K+dmLT
OmpC, Omplolb and Omp36K+LTAI+dmLT
OmpW, Omplolb and Omp36K+LTAI
OmpW, Omplolb and Omp36K+dmLT
OmpW, Omplolb and Omp36K+LTAI+dmLT

D. Methods of Treatment

The therapeutic methods of the disclosure (which include prophylactic treatment) in general include administration of a therapeutically effective amount of the compositions described herein to a subject in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, marker (as defined herein), family history, and the like).

In some aspects of the present disclosure, the present disclosure provides compounds which are administered without modification or administered as pro-drugs. In some embodiments, the compounds are administered in combination with another therapeutically agent wherein each agent is administered independently or wherein the drugs are combined through chemical modifications and a linker group. In some embodiments, the drugs are administered as a conjugate with a cell targeting moiety. In some embodiments, the compounds of the present disclosure are administered as a conjugate with an antibody.

E. Combination Therapies

It is very common in the field of medicine to combine therapeutic modalities. The following is a general discussion of therapies that may be used in conjunction with the vaccines of the present disclosure.

To treat infections using the methods and compositions of the present disclosure, one may contact a subject with vaccine and at least one other therapy. These therapies would be provided in a combined amount effective to achieve a reduction in one or more disease parameter. This process may involve contacting the bacteria/subject with the both agents/therapies at the same time, e.g., using a single composition or pharmacological formulation that includes both agents, or by contacting the bacteria/subject with two distinct compositions or formulations, at the same time, wherein one composition includes the vaccine and the other includes the other agent.

Alternatively, the vaccine may precede or follow the other treatment by intervals ranging from minutes to weeks. One would generally ensure that a significant period of time did not expire between the time of each delivery, such that the therapies would still be able to exert an advantageously combined effect on the cell/subject. In such instances, it is contemplated that one would contact the cell with both modalities within about 12-24 hours of each other, within about 6-12 hours of each other, or with a delay time of only about 12 hours. In some situations, it may be desirable to extend the time period for treatment significantly; however, where several days (2, 3, 4, 5, 6 or 7) to several weeks (1, 2, 3, 4, 5, 6, 7 or 8) lapse between the respective administrations.

It also is conceivable that more than one administration of either the vaccine or the other therapy will be desired. Various combinations may be employed, where a vaccine of the present disclosure is “A,” and the other therapy is “B,” as exemplified below:

| | | | | | | | | | | |
|---|---|---|---|---|---|---|---|---|---|---|
| A/B/A | B/A/B | B/B/A | A/A/B | B/A/A | A/B/B | B/B/B/A | B/B/A/B | A/A/B/B | A/B/A/B | A/B/B/A |
| B/B/A/A | B/A/B/A | B/A/A/B | B/B/B/A | A/A/A/B | B/A/A/A | A/B/A/A | A/A/B/A | A/B/B/B | B/A/B/B | B/B/A/B |

Other combinations are contemplated. The following is a general discussion of antibiotic therapies that may be used in combination with the vaccines of the present disclosure.

The term “antibiotics” are drugs which may be used to treat a bacterial infection through either inhibiting the growth of bacteria or killing bacteria. Without being bound by theory, it is believed that antibiotics can be classified into two major classes: bactericidal agents that kill bacteria or bacteriostatic agents that slow down or prevent the growth of bacteria.

In some embodiments, the present compounds are administered in combination with one or more additional antibiotic. In some embodiments, antibiotics can fall into a wide range of classes. In some embodiments, the compounds of the present disclosure may be used in conjunction with another antibiotic. In some embodiments, the compounds may be used in conjunction with a narrow spectrum antibiotic which targets a specific bacteria type. In some non-limiting examples of bactericidal antibiotics include penicillin, cephalosporin, polymyxin, rifamycin, lipiarmycin, quinolones, and sulfonamides. In some non-limiting examples of bacteriostatic antibiotics include macrolides, lincosamides, or tetracyclines. In some embodiments, the antibiotic is an aminoglycoside such as kanamycin and streptomycin, an ansamycin such as rifaximin and geldanamycin, a carbacephem such as loracarbef, a carbapenem such as ertapenem, imipenem, a cephalosporin such as cephalexin, cefixime, cefepime, and ceftobiprole, a glycopeptide such as vancomycin or teicoplanin, a lincosamide such as lincomycin and clindamycin, a lipopeptide such as daptomycin, a macrolide such as clarithromycin, spiramycin, azithromycin, and telithromycin, a monobactam such as aztreonam, a nitrofuran such as furazolidone and nitrofurantoin, an oxazolidonones such as linezolid, a penicillin such as amoxicillin, azlocillin, flucloxacillin, and penicillin G, an antibiotic polypeptide such as bacitracin, polymyxin B, and colistin, a quinolone such as ciprofloxacin, levofloxacin, and gatifloxacin, a sulfonamide such as silver sulfadiazine, mefenide, sulfadimethoxine, or sulfasalazine, or a tetracycline such as demeclocycline, doxycycline, minocycline, oxytetracycline, or tetracycline. In some embodiments, the compounds could be combined with a drug which acts against mycobacteria such as cycloserine, capreomycin, ethionamide, rifampicin, rifabutin, rifapentine, and streptomycin. Other antibiotics that are contemplated for combination therapies may include arsphenamine, chloramphenicol, fosfomycin, fusidic acid, metronidazole, mupirocin, platensimycin, quinupristin, dalfopristin, thiamphenicol, tigecycline, tinidazole, or trimethoprim.

### V. EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Materials and Methods

Immunization and Infection. The specified dose of Omp antigen was admixed with adjuvant and mice were immunized either subcutaneously or by mucosal intratracheal (i.t.) immunization in the lung under isoflurane anesthesia. Mice were immunized twice three weeks apart. To test vaccine efficacy, after the 2nd immunization, mice were then infected with 10⁴ live K. pneumoniae by oropharyngeal aspiration-tongue pull technique (referred as i.t.) and sacrificed at 24 hr after infection.

Mice and Intratracheal Administrations. C57BL/6 (B6), Ifng^{-/-}Ighm^{-/-} mice were purchased from the Jackson Laboratory or Taconic Farms and maintained in the Tulane vivarium. For bacterial challenge, 104 K1 K. pneumoniae (strain KP-396) were given to isoflurane anesthetized mice in sterile PBS (50 μl) by intratracheal inoculation. Mice were euthanized 24 hours later to assess bacterial burdens in the lung and spleen.

Experimental K. pneumoniae Infection. K. pneumoniae KP-396 serotype 1, was grown in 100 ml of tryptic soy broth (Difco) for 18 hr at 37° C. The quantity of 1 ml of the culture was added to 100 ml of fresh tryptic soy broth and grown for 2 hr, allowing the culture to reach early log phase. The concentration of K. pneumoniae was determined by measuring the absorbance at 600 nm. A standard curve of absorbance units based on known CFUs was used to calculate inoculum concentration. Bacteria were pelleted by centrifugation at 5,000 rpm for 15 min, washed twice in PBS, and resuspended at the desired concentration.

Statistical Analyses. Unpaired, two-tailed, Student's t tests, μ=0.05, were used to assess whether the means of two normally distributed groups differed significantly. One-way analysis of variance was used to compare multiple means. Significance is indicated as p<0.05, p<0.01, and p<0.001. All error bars represent the standard deviation (SD).

### Example 2—Results

Studies to date demonstrate that monovalent (OmpX) or quadravalent immunization with antigens derived from a K2 strain of K. pneumoniae in the lung affords significant protection to a heterologous challenge with a K1 strain of K. pneumoniae. The inventors believe this protection is due to the generation of cross-reactive antibodies and T cells. Thus, the described vaccines can be used to generate serotype independent vaccines against multiple capsular serotypes of K. pneumoniae. As evidence of cross reactive T-cells, vaccine efficacy is achieved in mice with genetic deletion of B cells.

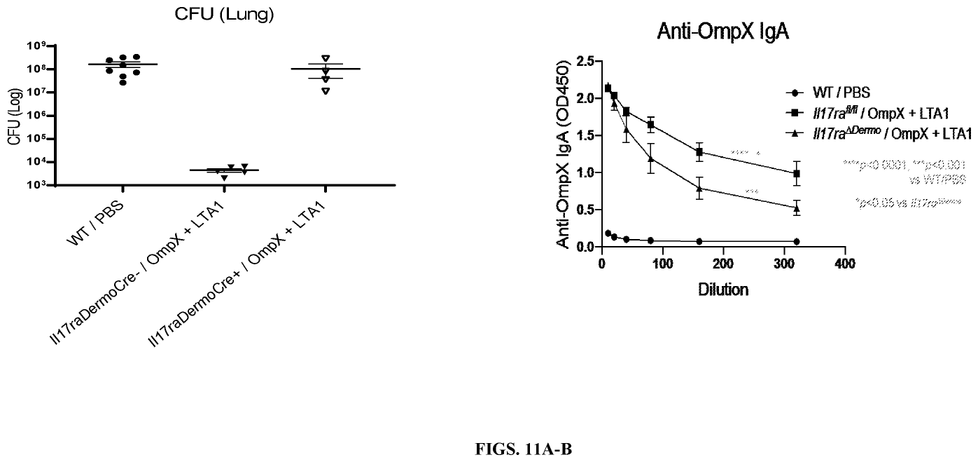

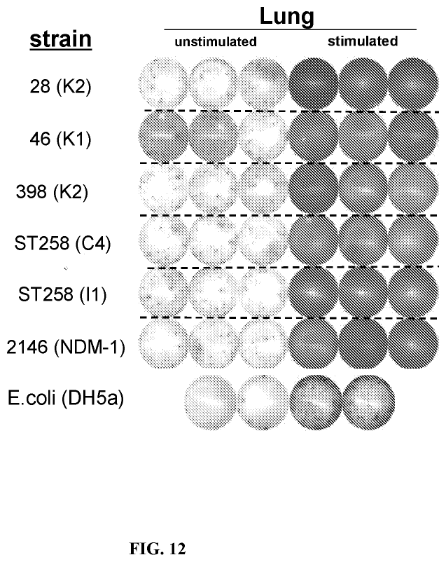

One of the challenges to develop mucosal vaccines that may rely on local B cell and T cell responses is that serum antibodies are not a reliable biomarker of mucosal responses. To identify protein biomarkers in the lung, the inventors deleted the gene encoding the receptor for IL-17RA in lung fibroblasts. Deletion of this receptor in lung fibroblasts significantly blocked vaccine efficacy (FIG. 11A) and was associated with a significant reduction in antigen specific IgA in the lung lavage fluid (FIG. 11B). Thus, the inventors believe that antigen specific IgA in lung/sputum may serve as an excellent biomarker of vaccine immunogenicity and protection.

Based on the conservation of OmpX in members of the Enterobacteriaceae family, the inventors predicted that immunization with Ompx from serotype 2 K. pneumoniae would elicit cross-reactive T cells with other K. pneumoniae serotypes as well as other members of the Enterobacteriaceae family such as E. coli. To test this, they immunized mice with OmpX+LTA1 adjuvant twice, three weeks apart and then assessed the ability of these cells to produce IL-17 in response ex vivo using an Elispot assay. As shown below, mice that were vaccinated with Ompx from K2 produced IL-17 (denoted by gray color) when stimulated with heat killed strains of K. pneumoniae including two carbapenem resistant strains (ST258 C4 and ST258 II) as well as E. coli. Thus this vaccine elicits immune responses to a broad class of gram negative pathogens.

Similarly, the inventors wanted to determine if Ompx elicits antibody (IgG) responses also cross reacted. Thus, they took serum from OmpX immunized mice and assessed whether serum IgG bound hypervirulent K1 strains of K. pneumoniae as well as ST258 multidrug resistant strains using surface detection of bound IgG using flow cytometry (FACS) analysis. FACS data clearly shows binding of OmpX anti-sera to the surface of these different serotypes of K. pneumoniae. Thus this antigen is accessible to humoral immunity independent of polysaccharide capsule.

### Example 3—Discussion

Most licensed vaccines for bacterial infections are effective by generating antibodies to polysaccharide capsules. A limitation of this approach is serotype replacement as encapsulated bacteria exist in the environment as multiple serotypes. Thus, there is a need for serotype independent approaches. By concentrating on conserved protein antigens expressed in the cell wall of bacteria, we have shown that specific outer membrane proteins elicit both B cell and T cell immunity that can provide heterologous immunity to other capsular serotypes. Moreover, based on the conservation of these antigens across the Enterobacteriaceae family, this work predicts that these vaccine elicited immune responses will protect against other related gram negative infections including those due to K. oxytoca, E. cloacae, and E. coli.

All of the compositions and/or methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and/or methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### VI. REFERENCES

The following references, to the extent that they provide exemplary procedural or other details supplementary to those set forth herein, are specifically incorporated herein by reference:
Remington's Pharmaceutical Sciences, 15th Edition.
Satline et al., Antimicrob. Agents Chemother. 61(4), 2017.
Satlin et al., Clin. Infect. Dis. 64(7)839-844, 2017.
Chen et al., Immunity 35(6):997-1000, 2011.

1. A composition comprising three or more outer membrane proteins (OMPs) from Klebsiella and at least one adjuvant dispersed in a pharmaceutically acceptable buffer, diluent or excipient.

2. The composition of claim 1, wherein the composition comprises three or more of OmpC, OmpW, Omplolb and Omp36K.

3. The composition of claim 1, wherein the composition comprises adjuvants selected from one or both of LTA1 and/or dmLT.

4. The composition of claim 1, wherein the adjuvant is linked to one or more of said OMPs.

5. The composition of claim 1, further comprising OmpX.

6. The composition of claim 1, wherein the composition is formulated for intranasal administration.

7. The composition of claim 1, wherein the composition is formulated for subcutaneous administration.

8. The vaccine of claim 1, wherein the composition is formulated for sublingual or intramuscular administration.

9. The composition of claim 1, wherein the composition comprises OmpC, OmpW, Omplolb, Omp36K, LTA1 and dmLT.

10. The composition of claim 1, wherein the composition comprises OmpC, OmpW, Omplolb, Omp36K, LTA1 and dmLT, but does not include any other OMPs or adjuvants.

11. A method of generating an immune response to a bacteria in a subject comprising administering to said subject a composition comprising three or more outer membrane proteins (OMPs) from Klebsiella and at least one adjuvant dispersed in a pharmaceutically acceptable buffer, diluent or excipient.

12. The method of claim 11, wherein the composition comprises three or more of OmpC, OmpW, Omplolb and Omp36K.

13. The method of claim 11, wherein the composition comprises adjuvants selected from one or both of LTA1 and/or dmLT.

14. The method of claim 11, wherein the adjuvant is linked to one or more of said OMPs.

15. The method of claim 11, wherein the composition further comprises OmpX.

16. The method of claim 11, wherein the composition is administered via intranasal administration.

17. The method of claim 11, wherein the composition is administered via subcutaneous administration.

18. The method of claim 11, wherein the composition is administered via sublingual or intramuscular administration.

19. The method of claim 11, wherein the composition comprises OmpC, OmpW, Omplolb, Omp36K, LTA1 and dmLT.

20. The method of claim 11, wherein the composition comprises OmpC, OmpW, Omplolb, Omp36K, LTA1 and dmLT, but does not include any other OMPs or adjuvants.

21. The method of claim 11, wherein the bacteria is Klebsiella.

22. The method of claim 21, wherein the bacteria is Klebsiella pneumoniae.

23. The method of claim 21, wherein the bacteria is Klebsiella oxytoca.

24. The method of claim 11, wherein the bacteria is Escherichia coli.

25. The method of claim 11, wherein the bacteria is Enterobacter cloacae.

26. The method of claim 11, wherein the bacteria is multi-drug resistant.

27. The method of claim 11, wherein the subject has a nosocomial bacterial infection.

28. The method of claim 11, wherein the subject has a post-surgical bacterial infection.

29. The method of claim 11, wherein the subject has a wound bacterial infection.

30. The method of claim 11, wherein the subject has a chronic or persistent bacterial infection.

31. A method of treating or preventing a bacteria infection in a subject comprising administering to said subject a composition comprising three or more outer membrane proteins (OMPs) from Klebsiella and at least one adjuvant dispersed in a pharmaceutically acceptable buffer, diluent or excipient.

32. The method of claim 31, wherein the composition comprises three or more of OmpC, OmpW, Omplolb and Omp36K.

33. The method of claim 31, wherein the composition comprises adjuvants selected from one or both of LTA1 and/or dmLT.

34. The method of claim 31, wherein the adjuvant is linked to one or more of said OMPs.

35. The method of claim 31, wherein the composition further comprises OmpX.

36. The method of claim 31, wherein the composition is administered via intranasal administration.

37. The method of claim 31, wherein the composition is administered via subcutaneous administration.

38. The method of claim 31, wherein the composition is administered via sublingual or intramuscular administration.

39. The method of claim 31, wherein the composition comprises OmpC, OmpW, Omplolb, Omp36K, LTA1 and dmLT.

40. The method of claim 31, wherein the composition comprises OmpC, OmpW, Omplolb, Omp36K, LTA1 and dmLT, but does not include any other OMPs or adjuvants.

41. The method of claim 31, wherein the bacterial infection is Klebsiella, such as Klebsiella pneumoniae or Klebsiella oxytoca.

42. The method of claim 31, wherein the bacterial infection is Escherichia coli.

43. The method of claim 31, wherein the bacterial infection is Enterobacter cloacae.

44. The method of claim 31, wherein the bacterial infection is multi-drug resistant.

45. The method of claim 31, wherein the subject has a nosocomial bacterial infection, a post-surgical bacterial infection, or a wound bacterial infection.

46. The method of claim 31, wherein the subject has a chronic or persistent bacterial infection.

47. The method of claim 31, further comprising treating said subject with another anti-bacterial therapy.

48. The method of claim 47, wherein said another anti-bacterial therapy is an antibiotic.

49. The method of claim 47, wherein said another anti-bacterial therapy is given before and/or after said composition.

50. The method of claim 47, wherein said another anti-bacterial therapy is concurrent with said composition.

51. The method of claim 31, wherein said subject is a human subject.

52. The method of claim 31, wherein said subject is a non-human mammal.

53. The method of claim 52, wherein said non-human mammal is a cow.

54. The method of claim 53, wherein said cow suffers from bovine mastitis.
